(19) United States
(12) Patent Application Publication
(10) Pub. No.: US 2020/0018767 A1
(43) Pub. Date: Jan. 16, 2020

(54) Methods of Making Organic Memristive/Memcapacitive Devices Induced Fermi Arc Surface States and Applications for Ultrasensitive Detecting Proteins and for Energy Harvesting Thereto

(71) Applicant: ELLEN TUANYING CHEN, ROCKVILLE, MD (US)

(72) Inventor: ELLEN TUANYING CHEN, ROCKVILLE, MD (US)

(21) Appl. No.: 16/512,356

(22) Filed: Jul. 15, 2019

Related U.S. Application Data
(60) Provisional application No. 62/698,107, filed on Jul. 14, 2018.

(51) Int. Cl. G01N 33/68; G01N 27/27; G01N 27/416; H01L 27/28

(52) U.S. Cl. CPC G01N 33/683; G01N 27/27; B82Y 15/00; H01L 27/285; G01N 27/4161

(57) ABSTRACT

A memristive/memcapacitive device with vertex double-helical polarized biomimetic protein nanotubules forming double membranes with potential gradient mimicking mitochondria's inner double membrane was invented. The memristive/memcapacitive device comprises a cross-linked conductive organic polymer having a single-wall cross-bar polarized nanotube self-assembling membrane (SAM) on a gold chip with a minimum 5 nm space between the nanotubes. Under an applied potential, a pair of vertex double-helical circular current flow induced the Fermi arcs states promoting a direct chelating with zinc ions of the Matrix Metalloproteinase (MMP-2), that made a dual-functioning direct ultrasensitive detection of protein in an attomolar concentration possible without a procedure of cycteine switch under label-free, probe-free and reagent-free conditions. The energy harvesting feature is also disclosed.

### CROSS-REFERENCE TO RELATED APPLICATIONS

This patent application is a US non-provisional patent application No. in title of that claims the benefit of U.S. Provisional Patent Application in titled of Organic Memristive/Memcapacitive Devices with a Polarized Single-Wall Nanotube Biomimetic Membrane Induce Fermi Arc Surface States for Sensing and Energy Harvesting Applications” Ser. No. 62/698,107 filed on Jul. 14, 2018 and also claim the benefit of the U.S. Non Provisional Patent Application in titled of Organic Nanobiomimetic Memristive/Memcapacitive Devices Ultrasensitive Direct Detect Matrix Metalloproteinase with Ser. No. 15/978,102 filed on May 12, 2018. The entire disclosure of the prior Patent Application Ser. Nos. 62/698,107 and 15/978,102 is hereby incorporated by reference, as is set forth herein in its entirety.

### FIELD OF THE INVENTION

The present invention relates to the field of electronic device, in particular, to a device having both characteristics in memristive/memcapacitive and Weyl semimetals for room-temperature switch current direction and making Fermi Arcs for Chelating with Matrix metalloproteinase under normal pressure and explore its applications in sensing and energy harvesting, such as for direct reagent-free sensing of attomolar concentration (aM) of Matrix Metalloproteinase (MMP) in biological specimens.

### BACKGROUND OF THE INVENTION

Matrix Metalloproteinase (MMP) is a family of zinc-dependent endopeptidases. The enzymes play a key role in human health for promoting newborn growth, nervous system growth, as well as in promoting various human diseases, such as cancer invasion, osteoarthritis, tissue destruction, diabetes, coronary malfunction, epilepsy and Alzheimer's [1-4]. MMP's major role is to degrade the extracellular matrix, and it is a double-edge sword. MMP-2 has been identified as a critical biomarker for diagnosing, monitoring and predicting multiple types of human diseases [4-9]. However, almost 50 MMP inhibitors failed in clinical trials due to lack of specificity of the inhibitor to MMP [10]. Improving sensor performance in the detection of MMPs in a sub fg/mL level among a wide dynamic range implemented with simplified procedures is a paramount challenge in the traditional enzyme-linked immunosorbent assays (ELISAs) method, labeling florescence method and the nanoparticle electrochemical sensing methods [11-12]. This is because most methods are subject to protein interference and time consuming, burdensome procedures that hamper reaching the goals. Our prior experiences in the development of nanostructured biomimetic sensors for direct detection of various biological biomarkers have encouraged us to seek an innovative approach and attempt to attack this problem for direct reagent-free detection of MMP-2 [13-18].

Development of polarized mimicking protein microtubules is an increasingly interesting subject in many nanoscale engineering applications [19]. However, a key challenge raised for direct detecting MMP-2 without using antibody, no reagent and no labeling is to focus on how to induce the direct biocommunication between the MMP-2 and the function groups in the sensor membrane when we decided to implement the biomimetic polarized protein microtubule pathway. Our approach is to build the artificial microtubules with cross-linked organic conductive polymers having multiple chelating imidazole ligands embedded. That enables the polymer ligands to have a strong affinity to coordinate with the zinc ions in the MMP-2. Plus the crossing-bar nanotubules might be favorable in the development of a nanostructured memcapacitive/memristive sensor for reagent-free, probe-free direct detecting of MMP-2.

Vertex double-helical bidirectional gas flowing patterns forming inside of a tornado are a known natural phenomenon. The bidirectional polarized circular current forming inside of a toroidal nanostructured memristive/memcapacitive sensing device in the presence of β-amyloidal (Aβ) in human blood specimen is akin to the tornado gases and caused severe voltage signal reduction at the 0.25 Hz Slow-wave Sleeping frequency (SWS) for monitoring of the neuronal circuitry integrity of Altheimers disease (AD) as reported [20-23]. Recent discoveries of Fermi arcs existing in either a Dirac semimetal and a Weyl semimetal provided evidence that a polarized electron gas formed a pair of massless Fermi arcs on a Dirac semimetal surface that are beneficial for a wide range of research in materials because of a unique electromagnetic property [24-26]. However, there were very few investigations, if any, to explore the possibility that a vertex double-helical current can be used as a candidate to explore the Fermi arc utilization in a memristive/memcapacitive device having an organic polymer comprised self-assembling membrane. One advantage of using the memcapacitive device is there is no heat release due to its nonlinear, bipolar, hysteresis nature of field dominating rather than thermo dominating [27]. The inspiring research reports encouraged our team to experimentally develop an organic cross-linked polymer memristive/memcapacitive toroidal device that forms a vertex double-helical circular current in a 3D architecture self-assembling membrane (SAM) in the presence of MMP-2, and to test our hypothesis: 1. the double-helical bidirectional direct electron-transfer (DET) circular current may create an environment of enriched electronegativity cloud for attracting zinc ion of MMP-2 based on the negative capacitance of the sensor membrane; 2. the chelating imidazole groups in the special derivative cyclodextrin toroidal cavity biomimetic cross-linked membrane may create an unique nanostructure that is not only able to mimic the structure of protein microtubules with cross-bar characteristics of the function of memristive/memcapacitive, but also is highly polarizable in an electrochemical field; 3. the vertex double-helical current may form direct electron-relay with zinc ions of MMP-2 that possess a Fermi arc state on the surface of the device membrane, and we may be able to see the 2D Fermi arc glow with nodes; and 4. the zinc ion chelating with the imidazole groups of the biomimetic membrane may change the bidirectional DET relay landscape.

### SUMMARY OF THE INVENTION

It is an object of the present invention to build the artificial microtubules with cross-linked organic conductive polymers having multiple chelating ligands embedded, that enable the polymer ligands to have a strong affinity to coordinate with the zinc ions in the MMP-2.

It is an object of the present invention to experimentally develop an organic cross-linked polymer memristive/memcapacitive toroidal device that forms a vertex double-helical circular current in a 3D architecture self-assembling membrane (SAM) in the presence of MMP-2,

It is an object of the present invention to develop a device forming the double-helical bidirectional direct electron-transfer (DET) circular current, that create an environment of enriched electronegativity cloud for attracting zinc ion of MMP-2 based on the negative capacitance of the sensor membrane.

It is an object of the present invention to develop a device chelating imidazole groups in the special derivative cyclodextrin toroidal cavity biomimetic cross-linked membrane creating the memristive/memcapacitive to be polarizable in an electrochemical field.

It is an object of the present invention to develop a device having the vertex double-helical current that possess a Fermi arc state on the surface of the device membrane, which we may be able to see the 2D and 3D Fermi arc glow with nodes present.

It is an object of the present invention to create an organic nanobiomimetic memristive/memcapacitive devices having sensing function for direct detection of protein molecules, and specifically for MMP without using a probe or denaturing the protein.

It is an object of the present invention to use the innovative memristive/memcapacitive sensors to quantitative detect MMP in attomolar concentration (aM) in a biological specimens without the presence of other protein interference.

It is an object of the present invention to detect MMPs in biological specimens in a 2-4 ms speed without sample preparation or treatment.

It is an object of the present invention to detect MMPs under conditions of reagent-free.

It is a further object of the present invention having the device able to be dual-functions, i.e., be a Chronoamperometric sensor and a voltage sensor.

It is a further object of the present invention to have a Detection of Limits (DOL) reaching orders of magnitude lower than published reports under antibody-free, tracer-free, and reagent-free conditions with simplified procedures.

### BRIEF DESCRIPTION OF THE DRAWINGS

FIG. 1A depicts the AFM 3D image for the device in a small 1 μm length scale. FIG. 1B is the cross-section analysis.

FIG. 2 depicts ordered cross nanotubes in a large 5 μm scale AFM image.

FIG. 3. depicts the art model for the proposed polarizable microtubule electron-relay system. The red dot refers to the imidazole receptors in the CD cavity with each cavity having 2 red dots.

FIG. 4 (L) Illustrates the hysteresis of the i-V curve of the memristor/memcapacitor in 40 ng/mL MMP-2 with consecutive scan cycles at 200 Hz scan rate. FIG. 4 (R) depicts the i-V curve in control solution at 200 Hz scan rate.

FIG. 5A, FIG. 5B and FIG. 5C represent the plots of normalized current of DET_{red}, DETₒₓ and the MEM peaks vs. 5 scan cycles, respectively.

FIG. 6A depicts the plot of energy density vs. applied potential in the control PBS solution in 5 consecutive scan at 200 Hz san rate. FIG. 6B depicts the plot of energy density vs. applied potential with 40 ng/mL MMP-2. FIG. 6C depicts the comparison of capacitance vs. applied potential between the control PBS and FIG. 6D depicts with 40 ng/mL MMP-2.

FIG. 7A depicts the 3D plot of the scattering data with dynamic relationship between real time (as Z axis), energy density (as X axis) and current (as Y axis) in the formation of vertex polarized double-helical current in biomimetic protein microtubule of the memristor/memcapacitor in pH 7.4 PBS with 200 Hz scan rate in 5 consecutive cycles. The red dots are the scatter raw data with each cycle has 3200 data points. FIG. 7B depicts the contour map showing the formation of the polarized double-helical current in a time scale increased as labeled by the number of scan cycles. FIG. 7C depicts the 3D plot of time impacts on energy change and current change in PBS solution. FIG. 7D depicts the 3D plot of the dynamic relationship between time, energy density and current in the formation of vertex double-helical current in the memristor/memcapacitor in the presence of 40 ng/mL MMP-2. FIG. 7E depicts the contour map change due to presence of MMP-2. FIG. 7F depicts the 3D plot of the MMP-2 impacts on energy distribution and current change in time between 0-80s. FIG. 7G depicts the 3D plot of the current in the scattering raw data (in red dots) of the vertex double-helical polarized nanotube membrane (as Z axis) and its trajectory orientation without MMP-2 in temporal time (as X axis) and spatial energy density (as Y axis), of the memristor/memcapacitor device in PBS solution with 200 Hz scan rate for 5 consecutive cycles. FIG. 7H depicts the 2D contour map plot of the polarized nanotube current's spatiotemporal orientation in an energy and real time landscape. FIG. 7I depicts the image of the double-helical current's relationship with its spatiotemporal orientation with the highest negative current expressed in the blue color vs. the positive current as the red color. Fermi arcs are shown as the bright arcs. FIG. 7J depicts the 3D plot of the current of the scattering raw data of the vertex double-helical polarized nanotube membrane and its trajectory orientation in temporal time and spatial energy density of the memristor/memcapacitor device in the presence of 40 ng/mL MMP-2 solution with 200 Hz scan rate for 5 consecutive cycles. FIG. 7K depicts the 2D contour map plot of the polarized nanotube current's spatiotemporal orientation in an energy and time landscape. FIG. 7L depicts the image change when MMP-2 was presented. FIG. 7M depicts the same 3D plot as of FIG. 7J. FIG. 7N depicts the 2D contour map plot using the same scale in time, energy density and current range as FIG. 7H in control for a fair comparison, respectively. FIG. 7O depicts the image change when MMP-2 was presented using the same scale in time, energy density and current range used as FIG. 7I in control, respectively.

FIG. 8A depicts the 3D plot of the double-helical current impacts on the absolute energy density (as Z axis), applied potential (as X axis) and capacitance distribution (as Y axis) in PBS control solution in 5 consecutive cycles with 200 Hz scan rate using the memristive/memcapacitive device. FIG. 8B depicts the contour map of the absolute energy density distribution due to the polarized double-helical current that impacts on capacitance and potential change. FIG. 8C is the energy image map related to potential and capacitance with the highest energy density as the dark color and the lowest energy density as the white color. FIG. 8D depicts the 3D plot of the double-helical current impacts on energy density, applied potential and capacitance distribution in the presence of 40 ng/mL MMP-2 solution using the memristive/memcapacitive device. FIG. 8E depicts the contour map change of MMP-2 impacts in energy density distribution on capacitance and potential change. FIG. 8F is the energy image map in the presence of MMP-2.

FIG. 9A depicts the 3D dynamic vertex double-helix current formation related in the DET_{red} and DETₒₓ peak's spatial location in the electric filed and the degree of the polarization ratio between DET_{red}/DETₒₓ, at 200 Hz scan rate in 10 consecutive cycles. FIG. 9B depicts the 2D contour map between current ratio, DET_{red} spatial location, DETₒₓ spatial location in applied electric field. FIG. 9C depicts the 3D plot of vertex surface DET ratio impact on the double-helical polarization in the PBS solution related to the change of DET_{red} and DETₒₓ peak's spatial location.

FIG. 10 depicts the memristive/memcapacitive sensor with polarized nanotube membrane acted as a energy harvesting device spontaneously discharge at 50 mA for 16.5 hours at room temperature in 1.3 mg/mL (mono substituted imidazole-β-dimetyl cyclodextrin) m-β-DMCD and 0.27 mM ZnCl₂ in 1M MeOH solution.

FIG. 11(A) depicts a plot of the current density vs. MMP-2 concentration over the range of 2.0×10⁻¹⁷ g/mL to 1.0×10⁻⁷ g/mL with triplicates. FIG. 11(B) depicts the CA curve profiles from 0.01 ng/mL to 100 ng/mL.

FIG. 12(A) depicts the CA curve profiles from 0.02 fg/mL to 40 fg/mL. The insert is the linear regression curve over the same MMP2 range. FIG. 12(B) depicts a linear calibration plot of the current density vs. MMP-2 concentration range from 0.01 ng/mL to 100 ng/mL with triplicates

FIG. 13 depicts the DSCPO voltage curves vs. time at 0.25 Hz at ±10A over 40 ag/mL to 100 ng/mL MMP-2 concentrations against the control samples with each sample run triplicates.

FIG. 14 depicts the volumetric energy density vs. MMP-2 concentrations.

FIG. 15 depicts the relationship between energy density of the sensor, MMP-2 concentration and specific capacitance using the voltage method.

### DETAILED DESCRIPTION OF THE INVENTION

### Example 1—Fabrication of the Nanostructured Self-Assembling Membrane (SAM) Gold Memristive/Memcapacitive Chips

The nanostructured biomimetic SAM was freshly prepared by forming cross linked conductive polymers from triacetyl-β-cyclodextrin (TCD), polyethylene glycol diglycidyl ether (PEG), poly(4-vinylpyridine) (PVP) and bis-substituted dimethyl-β-cyclodextrin (bM-β-DMCD) in a self-assembling manner on gold chips with appropriate proportions of the mixture. The polymer mixture was incubated at 80° C. for 2 hours before injecting it on the chip. After the injection, the chips were incubated for 96 hours at 37° C., then re incubated again for 2 hours after washing the chip with high purity water. The procedures of synthesis and characterization of bM-β-DMCD were based on the published literature [28]. MMP-2 enzyme was purchased from Ana Spec (Freemont, Calif.).

### Example 2—Characterization of the Biomimetic Microtubule Membrane

The morphology of the AU/SAM was characterized using an Atomic Force Microscope (AFM) (model Dimension Edge AFM, Bruker, Mass.). Data collected in TappingMode using silicon probes with 5-10 nm tip radius and ˜300 kHz resonance frequency (Probe mode TESPA-V2, Bruker, Mass.).

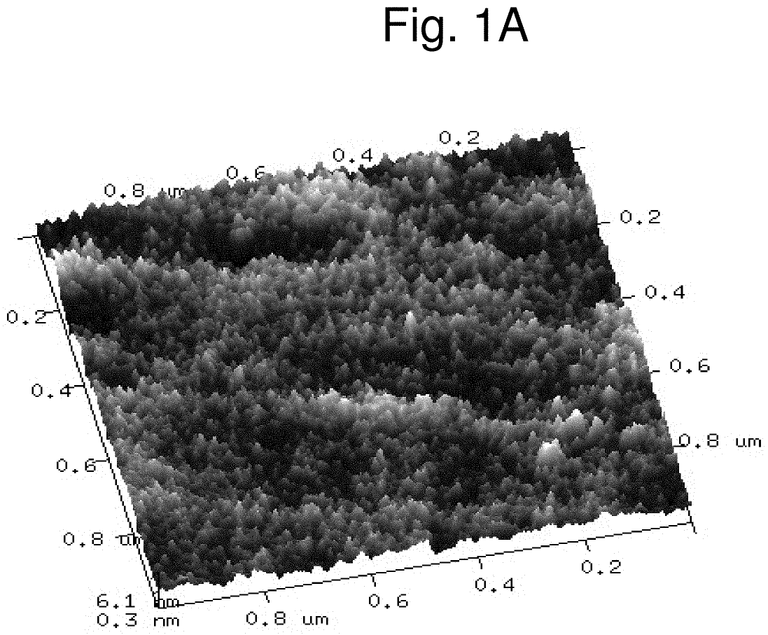

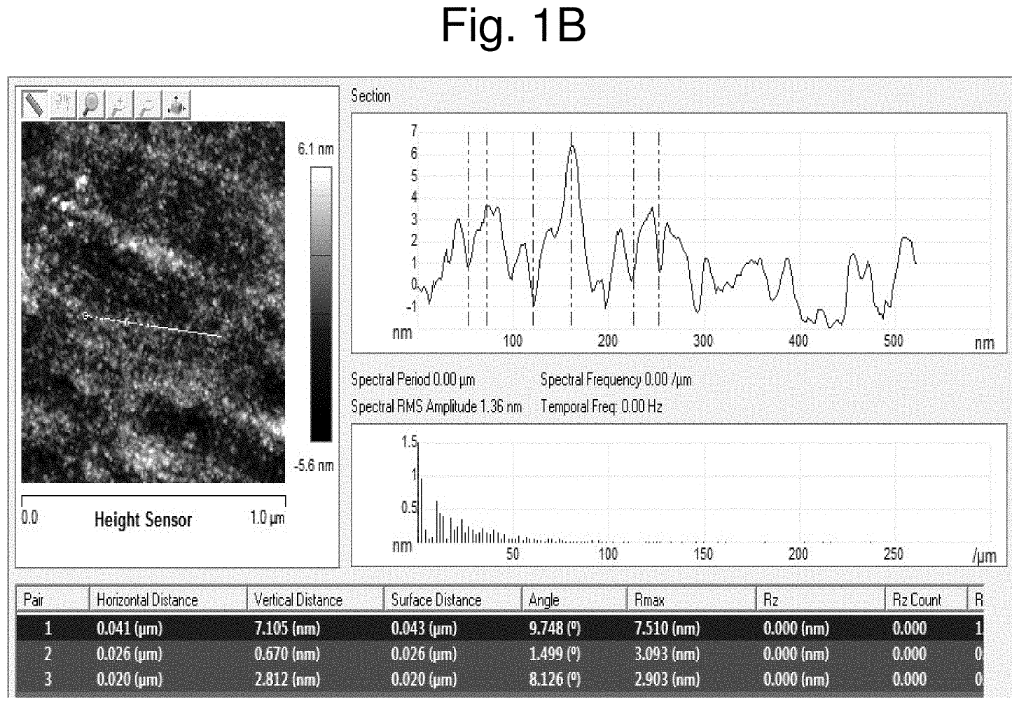

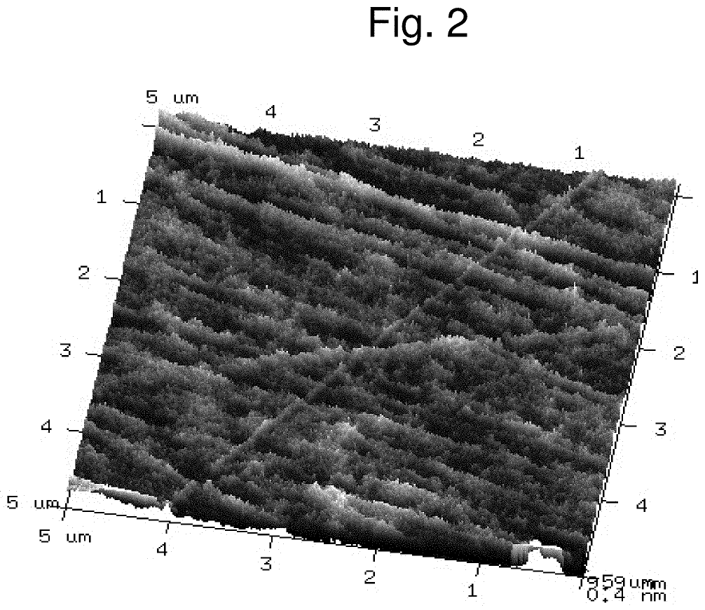

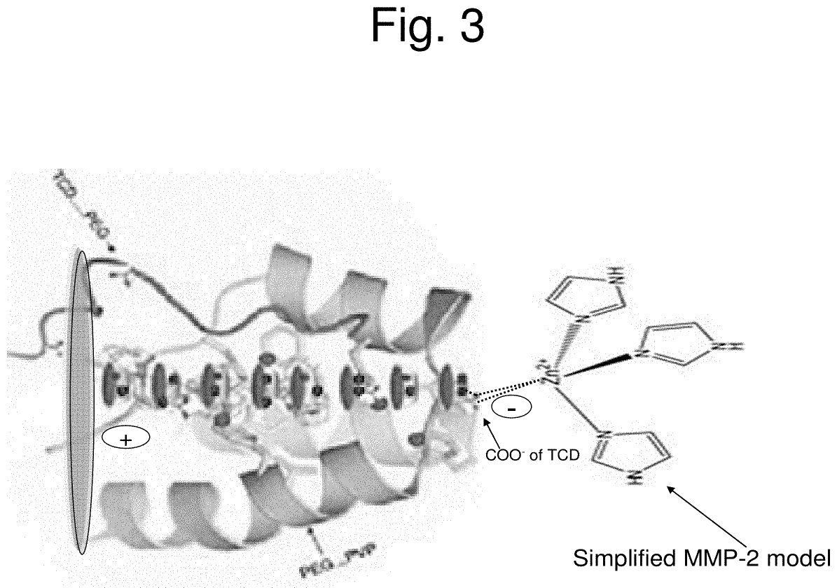

FIG. 1 illustrates the 3D structure of the membrane with an array of vertical nanopillars with crossing-bars in multiple layers in small scale with z value 15.1 nm, R_{q} 1.7 nm and Rₐ 1.3 nm. The amplitude channel shows changes in slopes and edges of features seen in height image, similar to a derivative of the Height channel. FIG. 2 shows the AFM well-ordered crossing-nanotube image in large scale. The proposed direct electro-transfer (DET) relay mechanism for detecting MMP-2 was depicted in an art model in FIG. 3. The right hand side is the simplified MMP model, and the induced direct bio-communication was shown through the zinc ion coordinating with both of the COO⁻ of TCD and the receptor groups of two imidazole in bm-β-DMCD cavity, i.e., by the coordination geometry, proton and electron transfers and the displacement of water molecules which formed the long electron-relay chain based on a favorable low ΔG [30-31]. The advantage of the sensor SAM is that it turns the inhibitory nature of the MMP-2 coordination complex as a potential cancer treatment drug into an agent for stimulating MMP-2 expression and forms polarizable microtubules shown from FIG. 1 to FIG. 3.

### Example 3—Evaluation of the Coordination Formation with Zinc Ions of MMP-2

Evaluations of the formation of a coordination complex between the MMP-2 and the ligands of the biomimetic membrane were based on a model mechanism proposed in FIG. 3. Two methods were used for the evaluations: (1) a cyclic voltammetry (CV) method was used to compare the dynamic rate constant results of direct electron-relay peaks and the MEM peaks vs. consecutive scan cycles (5) with or without MMP-2 at 200 mV/s scan rate at room temperature in pH 7.4 PBS solution. MMP-2 concentration is 40 ng/mL. (2) A comparison of Michaelis-Menten constant (kₘ) results using curves obtained from a chronoamperometric method (CA) with various MMP-2 concentrations from 2.0×10⁻¹⁷ g/mL to 1.0×10⁻⁷ g/mL compared with controls was described in the Section 2.5.

### Example 4—Comparing the Rate Constant by the CV Method

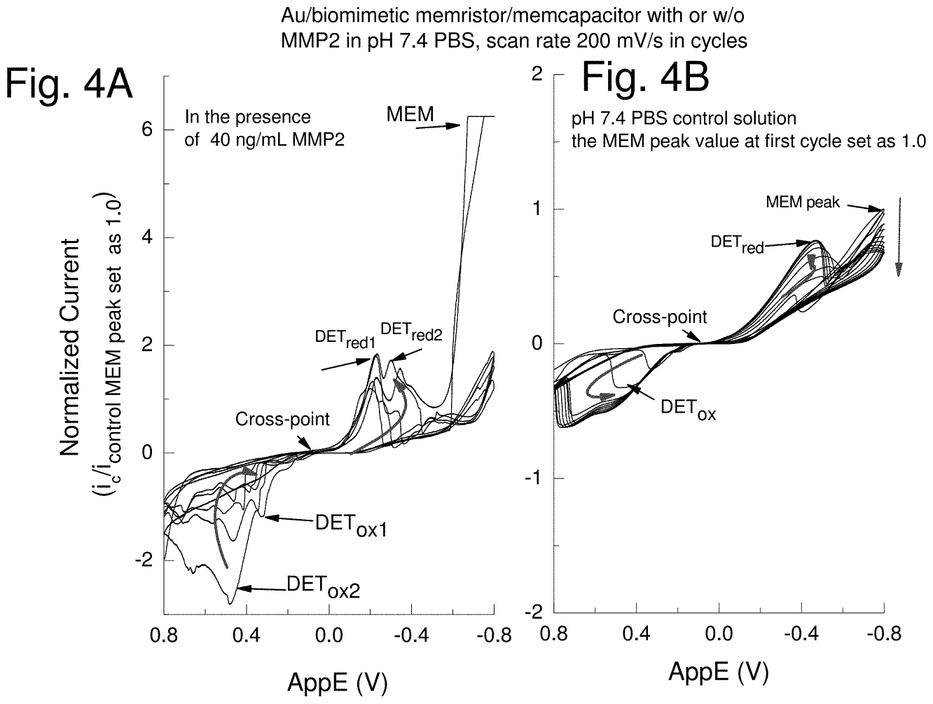

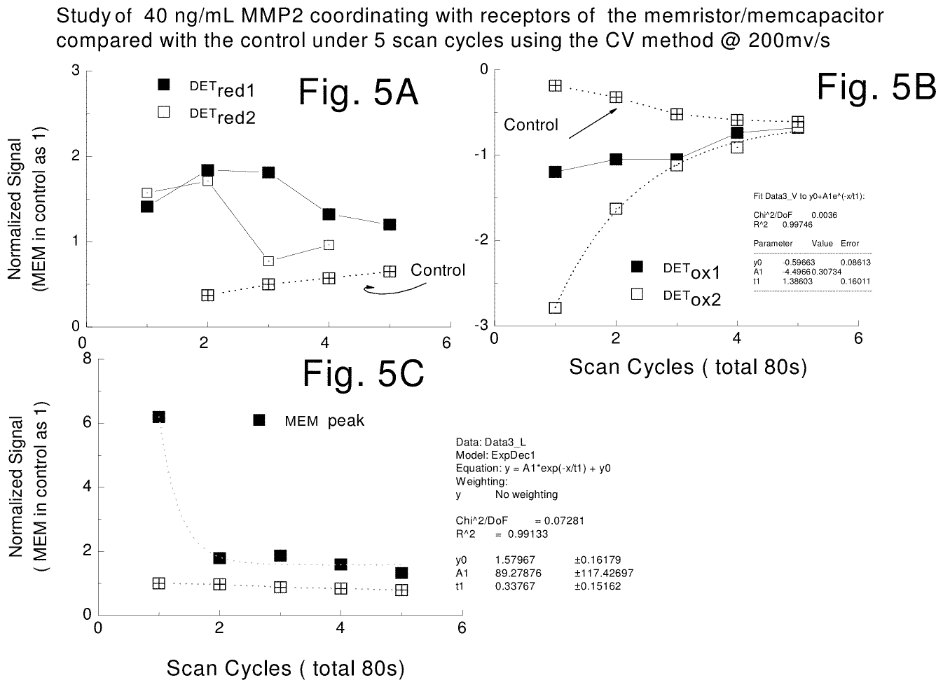

Memristor/memcapacitor exhibits not only hysteretic charge-voltage and capacitance-voltage curves but also negative and diverging capacitance within certain ranges of the field [32-33]. FIG. 4(R)'s i-V hysteresis curve is demonstrated with a switch point at the origin (0, 0) under consecutive scans at 200 Hz in PBS solution compared with that of having 40 ng/mL MMP-2 shown in FIG. 4(L). The increased positive and negative nonlinear potential movements of the DET_{red} and DETₒₓ peaks from the origin demonstrate there is bidirectional polarizable forces existing in the microtubules as the scan cycles increased. FIGS. 5A, B and C represent the plots of normalized current of DET_{red}, DETₒₓ and the MEM peaks vs. scan cycles, respectively. Table 1 compares the DET peak and the MEM peak rate constant results vs. scan cycles for with or without MMP-2. The results show the sensor interactions with MMP-2 have drastically broken the balanced, bidirectional, polarizable direct electron-relay and hole hopping into a more powerful asymmetric system. The rate constant and the amplitude of the MEM peak with MMP-2 increased 123 and 35.6-fold compared with the control, respectively. This demonstrates the charge of zinc ion coordinating with the gate ligand COO⁻ first at the sensor membrane with the rate constant and the amplitude of DETₒₓ₂ increased to 1.8 and 5.3-fold compared with the control indicating electron-relaying has taken place from COO⁻ to the first imidazole's proton transfer as the second step. Then the third step is the DET_{red1} peak with MMP-2, which increased the rate constant and the amplitude to 2 and 1.2-fold when coordinating with the second imidazole's free pair electrons compared with the control, respectively. Herein, directly detecting MMP-2 without using either an antibody or any other agents to activate MMP-2 in order to have a “cysteine switch” made possible, which is impossible according to conventional teaching [11-12, 34].

### Example 5—Comparing the Kₘ Constant

For comparing the Km results of the ligands of the sensor membrane affiliated with the MMP-2, Lineweaver-Burke plots were constructed. The Km value is 6.75 pM over 7.0×10⁻¹³ to 1.4×10⁻⁹ M, which is orders of magnitude stronger complexation than reported MMP-2's Km value for type 1 collagen of 8.5 μM [35-36]. The MMP-2 concentration is between 2×10⁻¹⁷ to 8.0×10⁻¹⁶M, K_{c} value is 1.6×10⁷/s and the K_{c}/Kₘ=6.4×10¹⁸ s⁻¹·M⁻¹.

### Example 6—Comparing MMP-2 Affect on Energy Density and Capacitance Using the CV Method

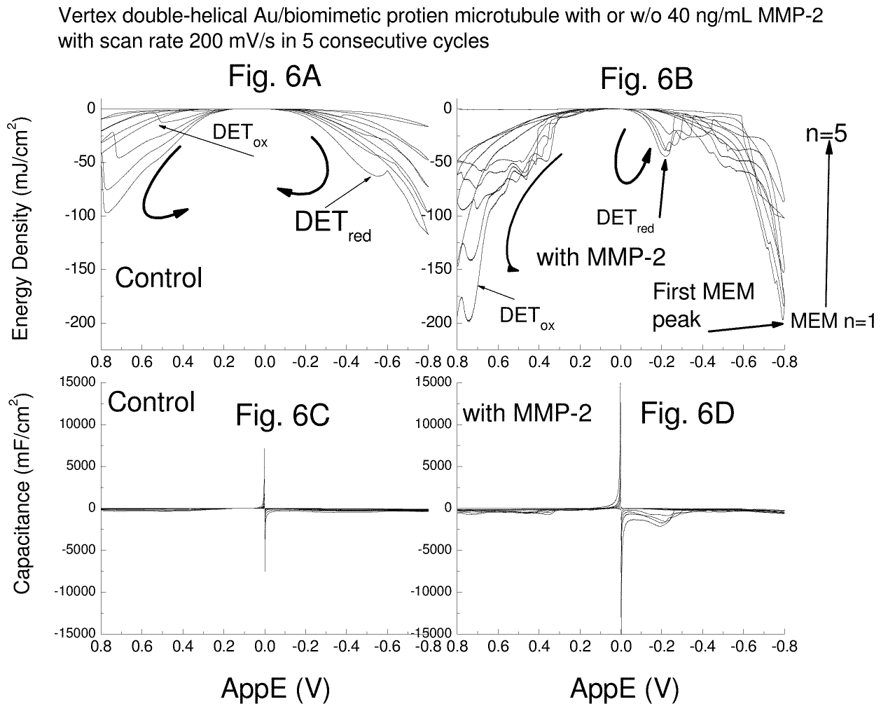

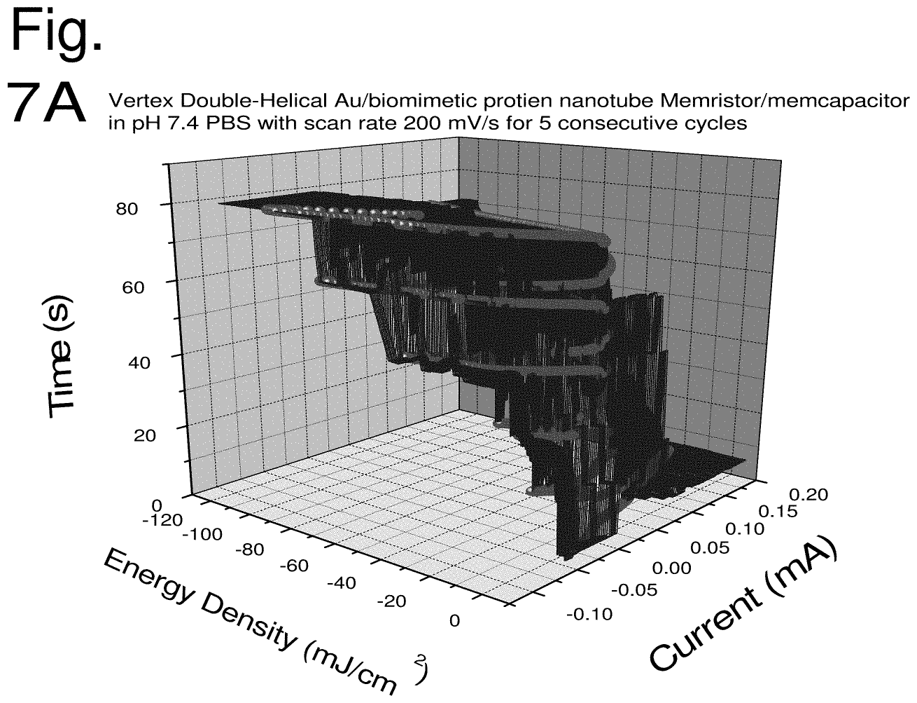

Results used for comparing MMP-2 affecting the negative energy density were presented in FIG. 6B and FIG. 6D against the controls of FIG. 6A and FIG. 6C in three scenarios in respect to (1) the DETₒₓ peak played the role and (2) the DET_{red} peak played the role and (3) the MEM peak played a role concerning energy density and capacitance in the electrochemical potential range we studied at a fixed scan rate. FIG. 6B demonstrates the DETₒₓ peak intensity with 2-fold increased negative energy density in the presence of 40 ng/mL MMP-2 at the 5^{th} scan cycle compared with that of the control in FIG. 6A. We noticed that the significant change of energy density on MEM peak at the first scan cycle was more than a 20-fold increase for with MMP-2 than the control, where it indicates the zinc ions of MMP-2 chelating well with the COO⁻ of TCD and the two imidazole groups in the cavity of bM-β-DMCD addressed in Section 3.2.1. We also noticed that the DETₒₓ has more impact on the negative energy density with MMP-2 compared with that of DET_{red} on the negative energy density without MMP-2.

Results used for comparing MMP-2's affects on the specific capacitance of the memcapacitor were presented in FIG. 6C and FIG. 6D. FIG. 6D demonstrates that the symmetric positive and the negative capacitance peaks' values at 0.0 V (absence of an electric field) increased 3.6-fold due to the presence of MMP-2 compared to without MMP-2. The DET_{red} peak only exists at −0.2V in FIG. 6D, and played a role having increased negative capacitance 8-fold when compared to conditions without MMP-2 in FIG. 6C [33].

The hysteresis behaviors are demonstrated by the memristive/memcapacitive device in both with and without MMP-2 in the 5 scan cycles with the cross-points at zero electrochemical potential field and zero energy density.

### Example 7—Evaluation of the Vertex Double-Helical Circular Current and its Induction of a Fermi Arc Surface State

Evaluation of the vertex double-helical circular current and its induction of Fermi arc surface state were based on a 3D dynamic mapping method with scatter raw data and their trajectory in (1) the time of 5 consecutive scans from 0 to 80s (as Z axis), the energy density (as X axis) and the current (as Y axis) was used for evaluation of the vertex double-helical current formation with or without MMP-2; (2). the current (as Z axis), the temporal time (as X axis) and the spatial energy density (as Y axis) was used for evaluation of the Fermi arc formation with or without MMP-2; (3). the absolute energy density (as Z axis), the applied potential (as X axis) and the capacitance distribution (as Y axis) was used for evaluation of the existence of a pair of Dirac Cones. (4). the current ratio of DET_{red} vs, DETₒₓ peak's intensity (as Z axis), the DET_{red} peak's spatial location in the electric filed (in eV as X axis) and DETₒₓ spatial location in applied electric field (in eV as Y axis) was used for evaluation of the existence of a spin berry phase without MMP-2 at 10 consecutive scan cycles from 0 to 160 s at 200 mV/s scan rate. In each of the four cases, data were presented in three categories: a plot of 3D raw data in trajectory, a 2D contour map plot and either an optical image or a 3D plot.

### Example 8—Single-Wall Cross-Bar Nanotubule Membrane Induces Double-Helical Polarized Circular Current (CC)

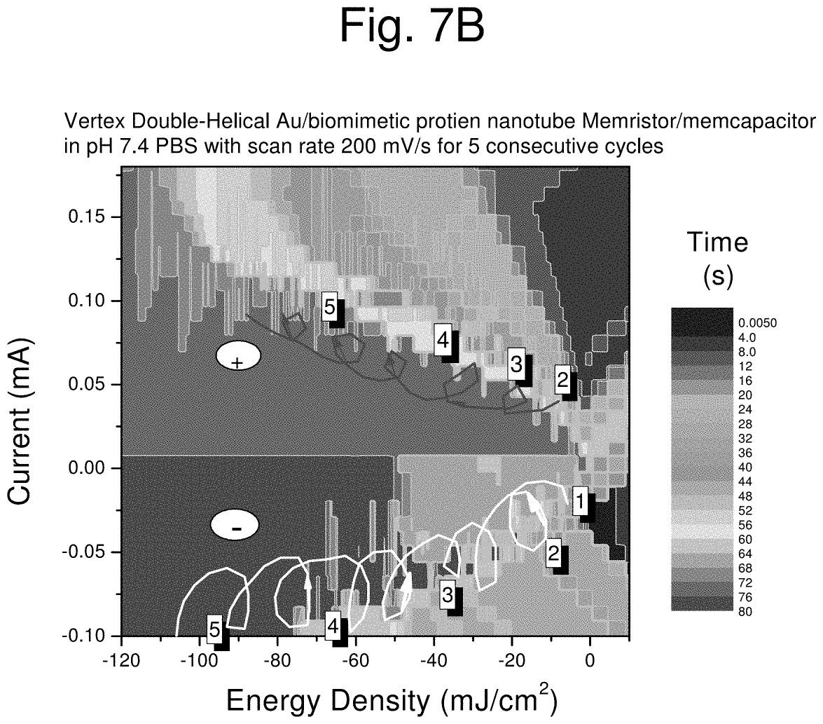

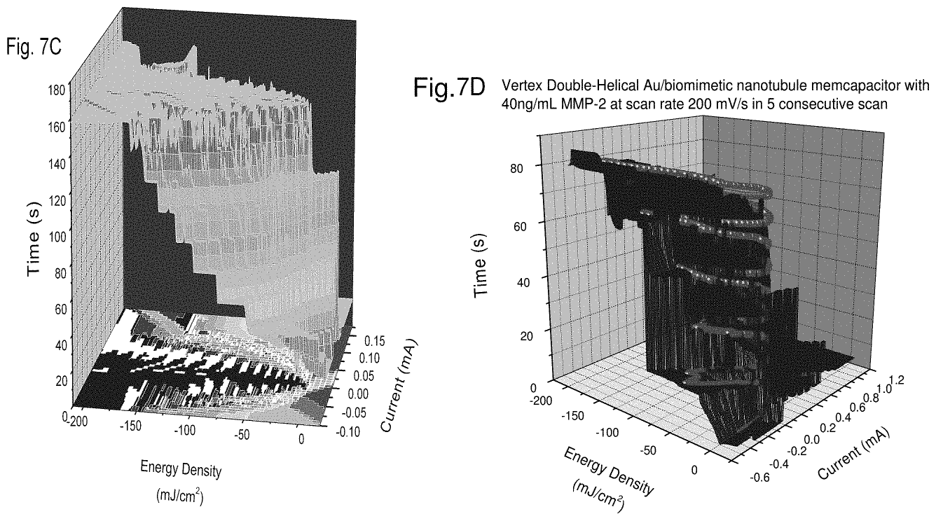

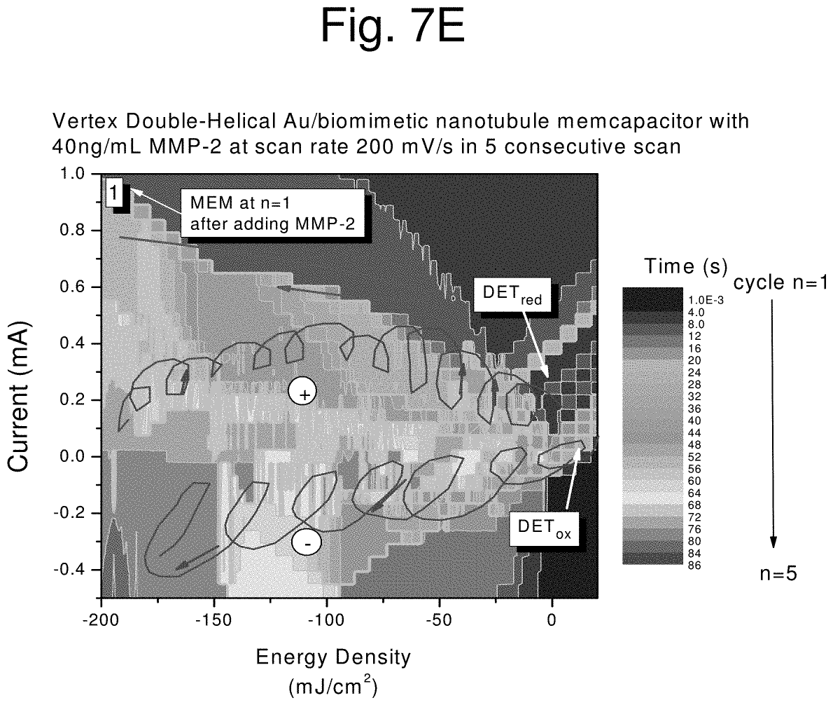

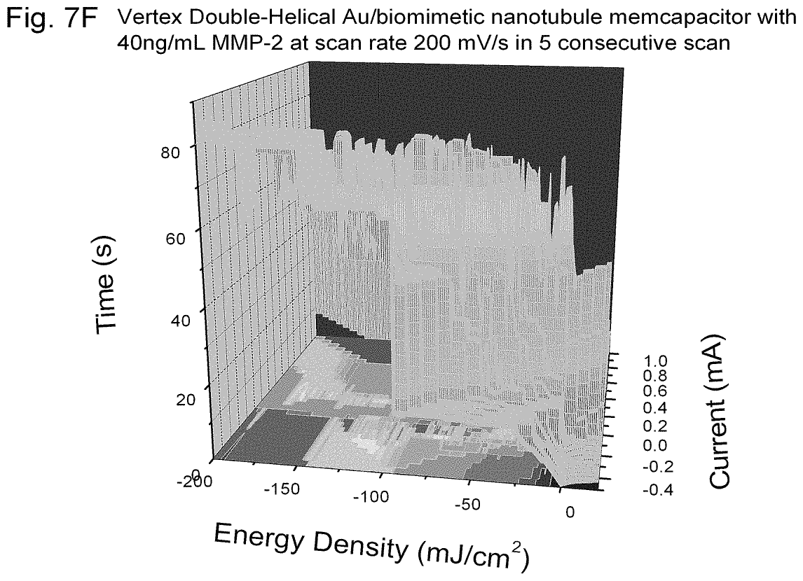

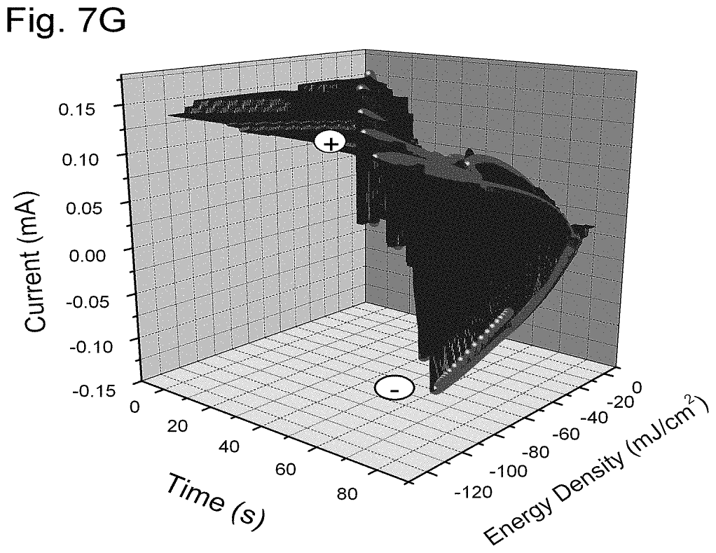

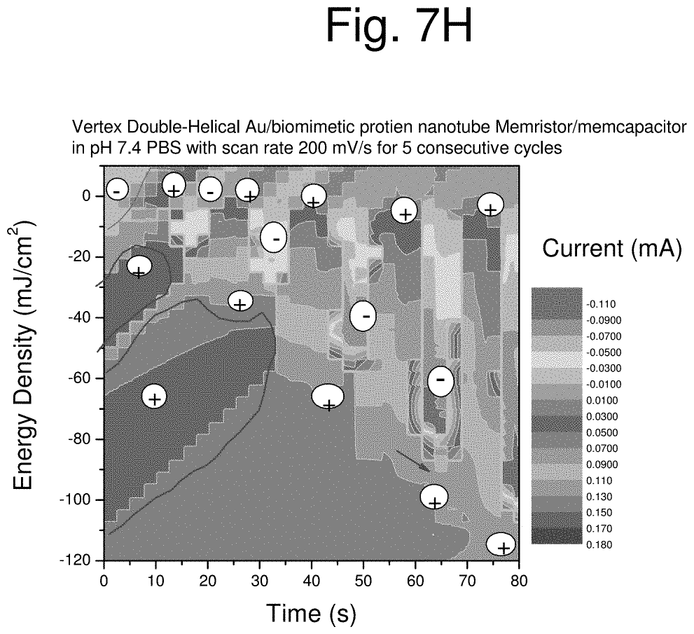

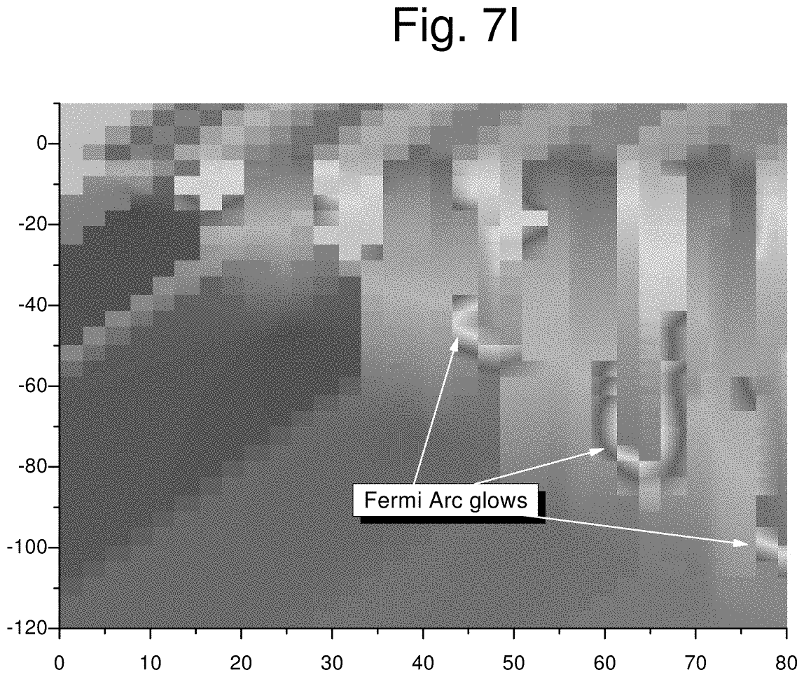

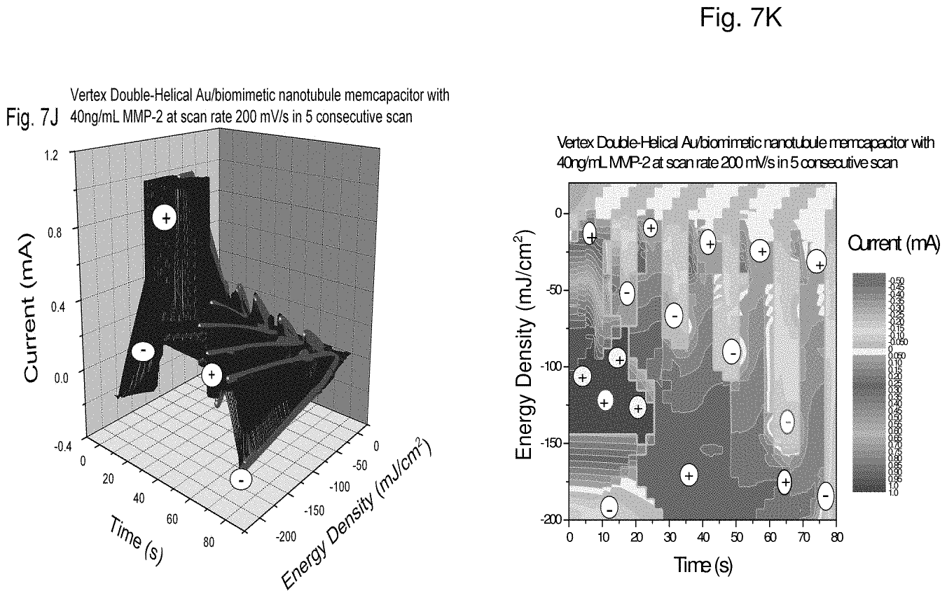

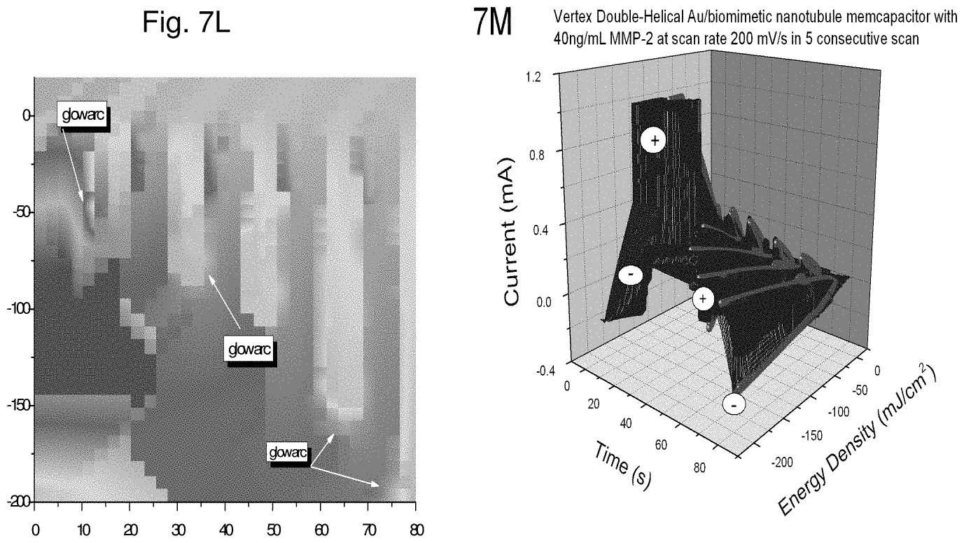

Circular current induced by junctions of aromatic molecules of the delocalized molecules has drawn interest among theoretical scientists [37-38]. Scientists have envisioned its future applications [37-38]. We reported the first observation of a biomimetic ACH sensor device having the electron-relay circular current phenomena within its applications in cancer spatio-temporal orientation, energy storage, and energy harvesting [18-39]. FIG. 1 and FIG. 2 demonstrated the AFM images of the single-wall cross-bar nanotube multiple-layer membrane, and its polarized current is depicted in FIG. 4; the results are presented in Table 1. Nevertheless, we still need to know the 3D trajectory orientation of the polarized nanotube circular current relationship with energy density and the scan time in the 5 scan cycles both with and without MMP-2. FIGS. 7A, 7B, and 7C depict the vertex double-helical polarized circular current in a 3D dynamic spatio-temporal orientation in the form of scatter plot raw data, a contour map, and a 3D color plot without MMP-2, respectively, compared with that of 40 ng/mL MMP-2 in FIGS. 7D, 7E and 7F, respectively. We found the vertex double-helical current formed in both cases, but the DET_{red} and the DETₒₓ equally influenced the energy density in conditions without MMP-2 at higher scan cycles than at lower cycle numbers in FIG. 7B; as compared with that of having MMP-2, where the DET_{red} and DETₒₓ have increased the negative energy density in the ratio of 1:2 in FIG. 7E. For conditions with MMP-2, the positive current increased more than 5-fold compared with those without MMP-2, and including a 2-fold reduction of negative current. Because of the polarization of the double-helical nanotube current initiated in the biomimetic protein microtubules, we saw a double-polarized membrane potential gradient exists in FIG. 7B and in FIG. 7E, that mimicked the mitochondria's inner double-layer membrane gradient electrochemical potential [40].

### Example 9—Observation of the Fermi Arcs

Researchers reported Fermi arcs existing in either a Dirac semimetal and a Weyl semimetal with evidence that a polarized electron gas formed a pair of massless Fermi arcs on a Dirac semimetal surface that are beneficial to its unique electromagnetic property for a wide range of research applications in material field. We know the vertex double-helical circular current has different type of charge at the two ends of the nanotubes, hence we expect to see the Fermi arc contour surfaces as we constructed the 3D plots that show the polarized nanotube circular current in a close relationship with the energy density and the scan time, in 5 scan cycles in two cases for with or without MMP-2 as shown in FIGS. 7G, 7H, and 7I for without MMP-2 compared with MMP-2 shown in FIGS. 7J, 7K and 7L, respectively. The image of Fermi arcs with nodes can be seen in image FIG. 7I located in higher scan cycles along the diagonal surface between energy density and time; the multiple layer double membrane in potential gradient is shown at the early scan cycles, and the positive charge was a dominate contributor to the negative energy density compared with that of negative charge contribution in FIG. 7H. The overall area ratio of positive charge/negative charge among the time from 0 to 80s is symmetric. In contrast, for with MMP-2, the Fermi arcs strength was reduced due to the negative charge increase seen in FIG. 7L with the energy density scale between 0 to −200 mJ/cm² and the area ratio of positive charge/negative charge among the time from 0 to 80s that contributed to the negative energy density is asymmetric, except at the first scan cycle, zinc ion chelating with the COO⁻ of TCD outside of the toroidal array of the membrane increased the positive charge contribution to the negative energy density shown in FIG. 4L and FIG. 7E of MEM peak's extreme high positive current. The 3D scatter raw data shown in FIG. 7J consists of a tall positive current “chimney column” at first scan cycle and a “curvature roof” of polarized positive and negative current along sitting on a surface of the 80s in the total 5 cycles scan time and the negative energy density up to −200 mJ/cm² with the “back roof gutter” has the band of zero energy density, which is different from FIG. 7G in a “Hummingbird” fly “8” shape with 10% of the positive current strength compared with FIG. 7J and 40% of the negative current strength compared with FIG. 7J. FIG. 7M depicts the same 3D plot as of in FIG. 7J. FIG. 7N depicts the 2D contour map plot using the same scale in time, energy density and current range used in FIG. 7H in control for a fair comparison, respectively. FIG. 7O depicts the image change when MMP-2 was presented under the same scale range compared with the control as FIG. 7I, respectively. One can see the double-helical CC induced more strong Fermi arcs covered all the scan cycles in the presence of MMP-2 even at the first cycle compared with the control of FIG. 7I, because at the energy density range between −10 to −120 mJ/cm², positive current and negative current were increased drastically for the case of with MMP-2 compared without MMP-2.

### Example 10—Absolute Energy Density Impact on Potential and Capacitance

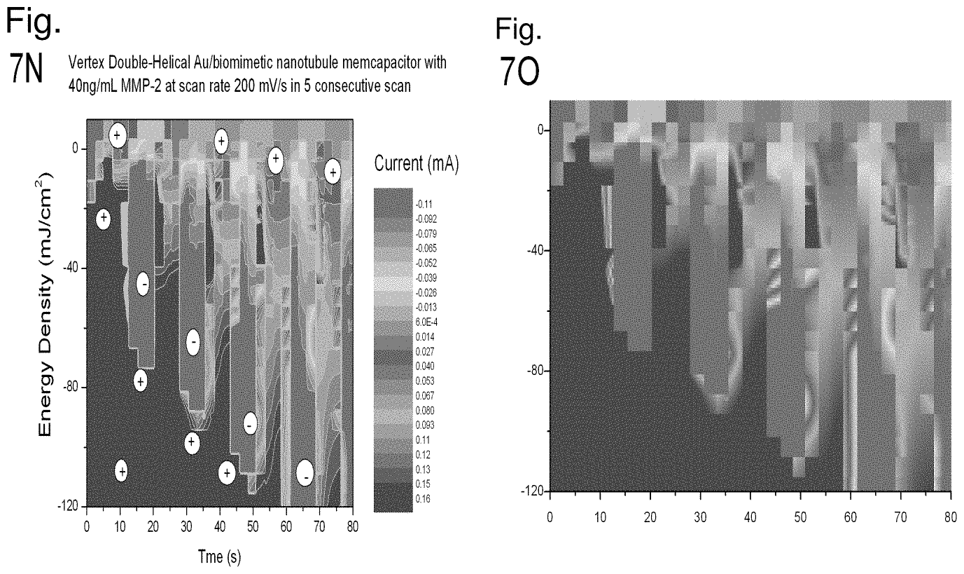

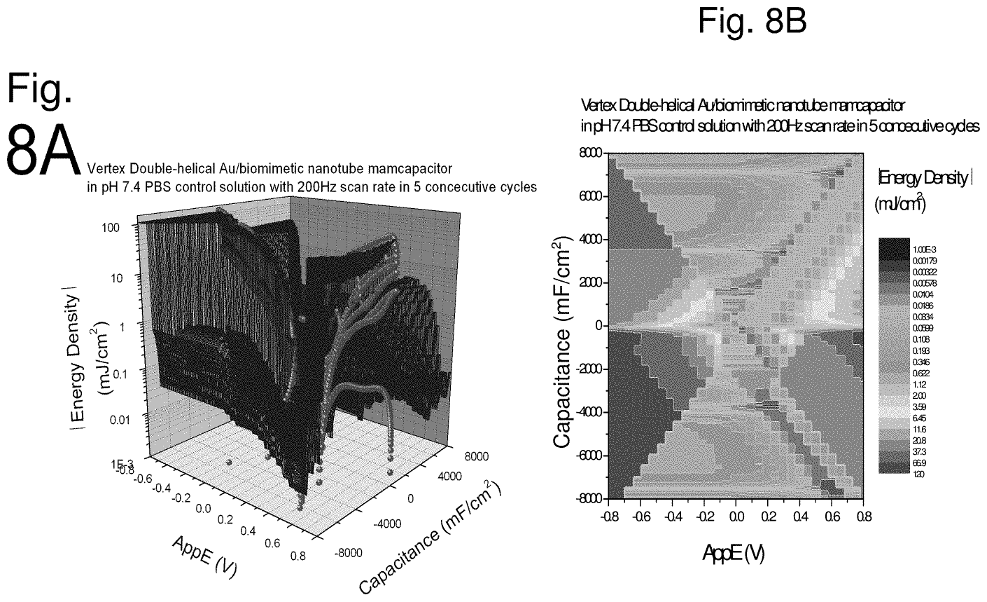

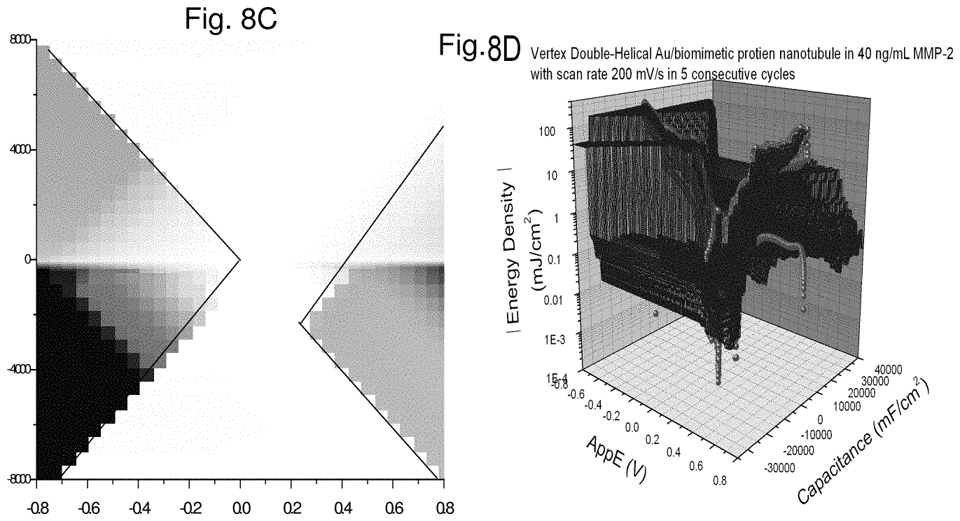

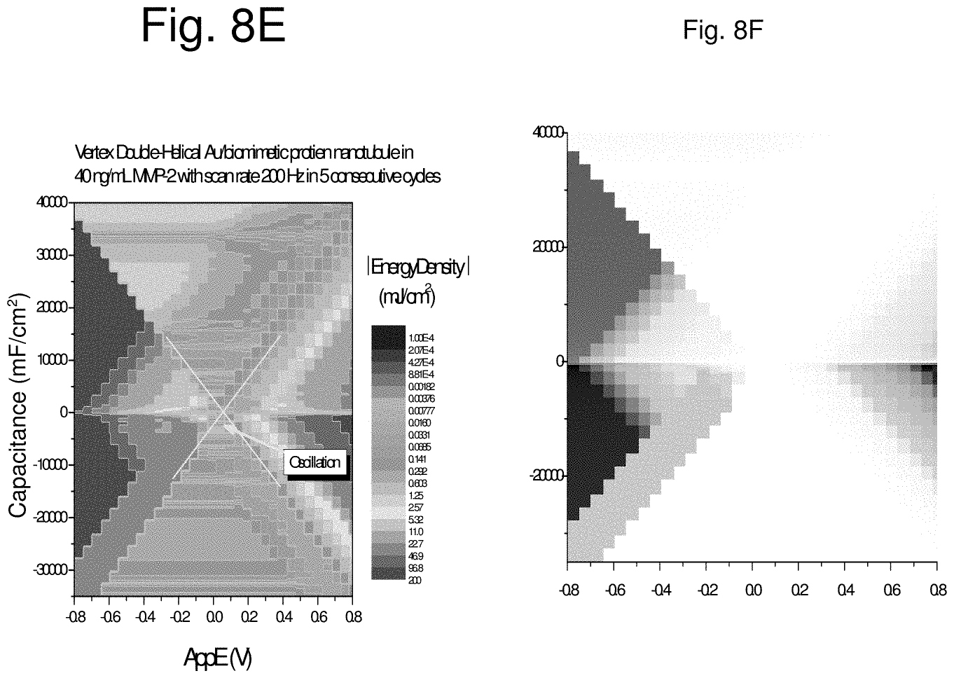

Absolute energy density impact on capacitance and potential was evaluated by the 3D mapping method and presented in FIGS. 8A, 8B and 8C for without MMP-2 compared with FIGS. 8D, 8E and 8F for with 40 ng/mL MMP-2, respectively. The overall 4.4-fold higher positive and negative capacitance contributed to the increase of the absolute energy density for with MMP-2 shown in FIG. 8E and FIG. 8F compared without MMP-2. We see the perfect alignment of the pair of Dirac cones with 1D gapless helical lines in FIG. 8E contour map with the Dirac node in the center having oscillation at the same spin direction on both cone surfaces compared with FIG. 8B, that the two cones are miss alignment. We are expecting the memristive/memcapacitive device with MMP-2 will be a more powerful device in energy harvesting and sensing purposes.

### Example 11—Observation of the “Spin Berry Phase”

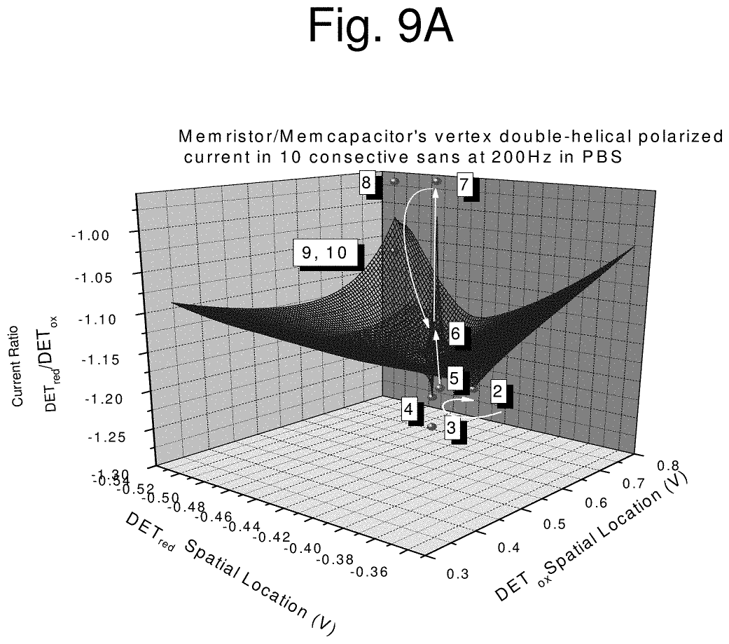

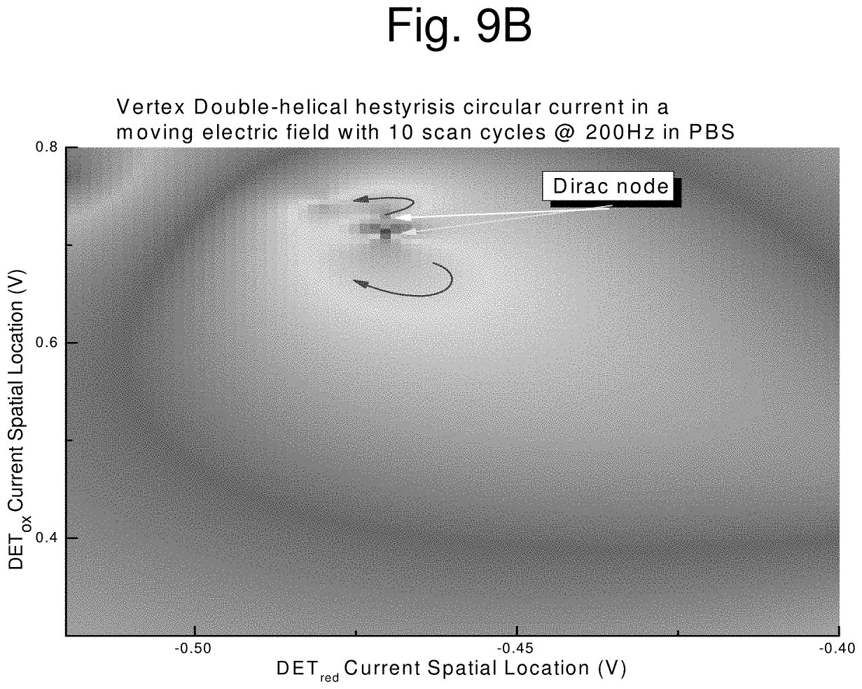

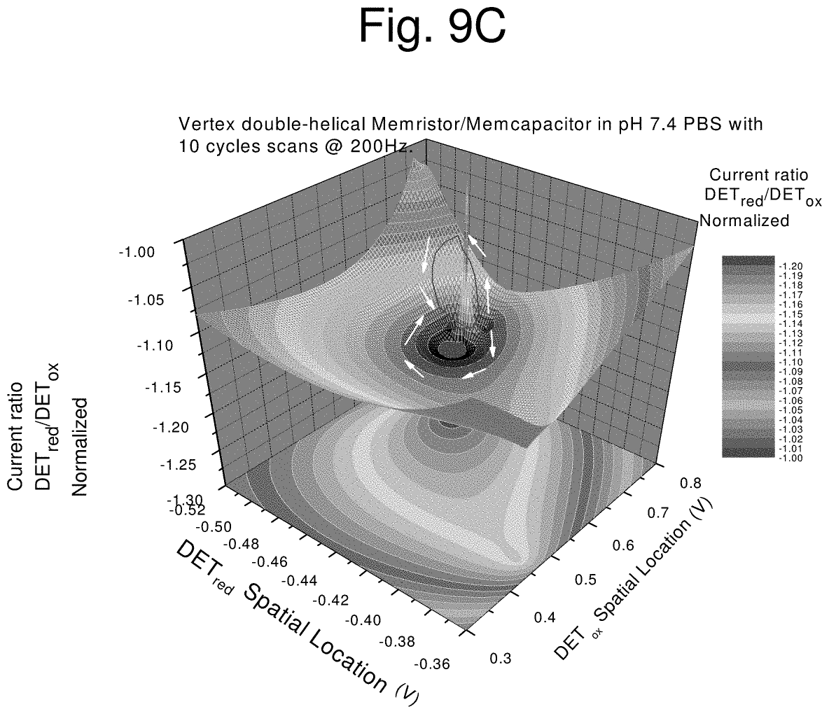

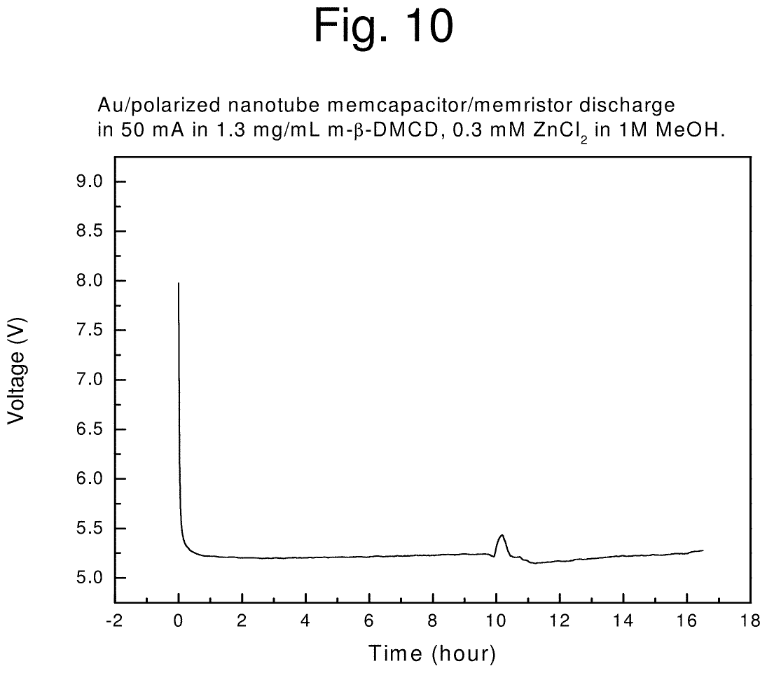

The “Spin Berry Phase” has been known for its characteristics in topological insulators and Weyl semimetals [24-26]. Results presented in FIG. 9A depicts the 3D dynamic spin berry phase formation expressed in the DET_{red} and DETₒₓ peak's spatial location in the electrochemical field and the degree of the polarization current ratio of DET_{red}/DETₒₓ, at 200 Hz scan rate in 10 consecutive cycles. FIG. 9B is the contour map and FIG. 9C is the 3D Berry Phase plot with different current flow directions without MMP-2.

### Example 12—Experimental Conditions for Quantitation of MMP-2

Quantitation of MMP-2 was conducted in two methods: the CA method and the Double Step Chronopotentiometry (DSCPO) method. The data were acquired at room temperature under fixed applied potentials for the CA method with 4 MHz data rate in MMP-2 final concentrations ranging from 2.0×10⁻¹⁷ g/mL to 1.0×10⁻⁷ g/mL with triplicates compared with pH 7.4 PBS controls. Curves presented were after taken an absolution for better visualization. Fixed ±10 nA and 4s step time was used with 1 KHz data rate for the DSCPO method with similar MMP-2 concentration ranges with samples run triplicate. MMP-2 samples were freshly prepared. Before the measurements, the standards samples were incubated at 37° C. for 2 hours. The preliminary applications were to detect the MMP-2 activities present in the NIST SRM 965A reference human serum samples with known hypo-, normal and hyperglycemia concentrations, respectively. An electrochemical work station was used (Epsilon, BASi, IN) with a software package from BASi. Origin Pro 2016 (Origin Lab Corp., MA) was used for all statistical data analysis and figure plotting.

### Example 13—Results in the Quantitation of MMP-2 by the CA Method

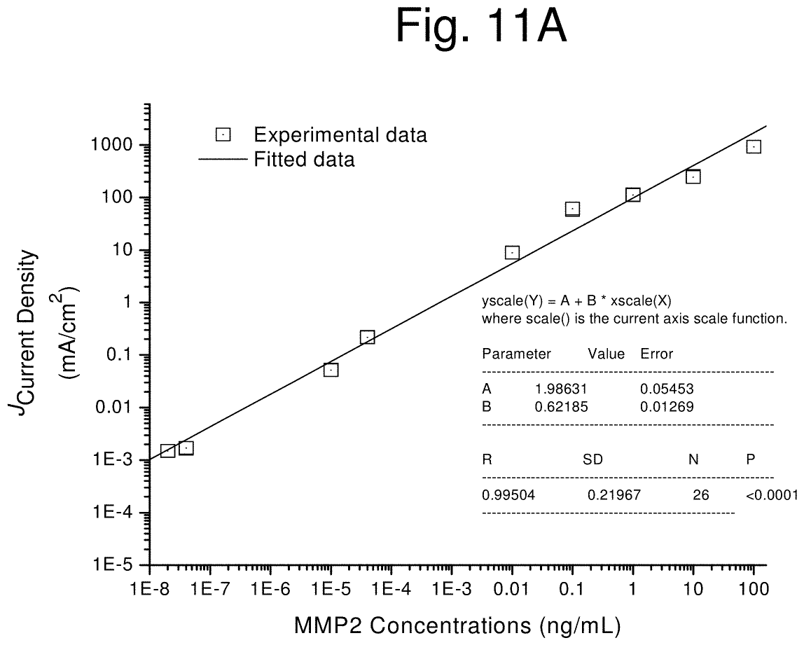

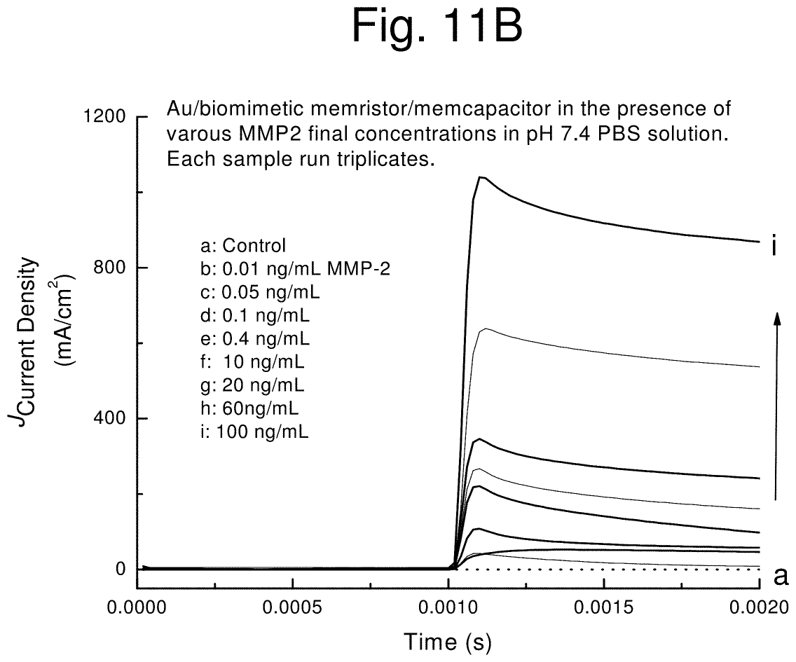

FIG. 11(A) depicts a double-log plot of current density vs. MMP-2 concentration over the range of 0.02 fg/mL to 100 ng/mL. The CA method has a Detection of Limits (DOL) value of 8.67×10⁻¹⁸ g/mL in PBS solution related to current density between 1.47 μA/cm² and 919.2 mA/cm² over MMP2 concentration between 20 ag/mL to 100 ng/mL with a Relative Pooled Standard Deviation 1.4% (n=26). FIG. 11(B) depicts the CA curve profiles from 0.01 ng/mL to 100 ng/mL.

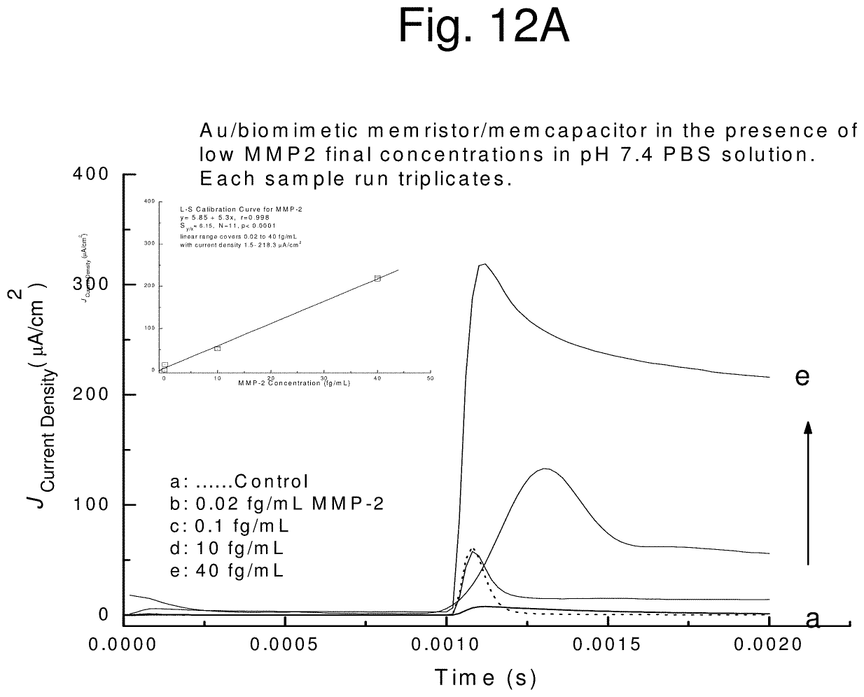

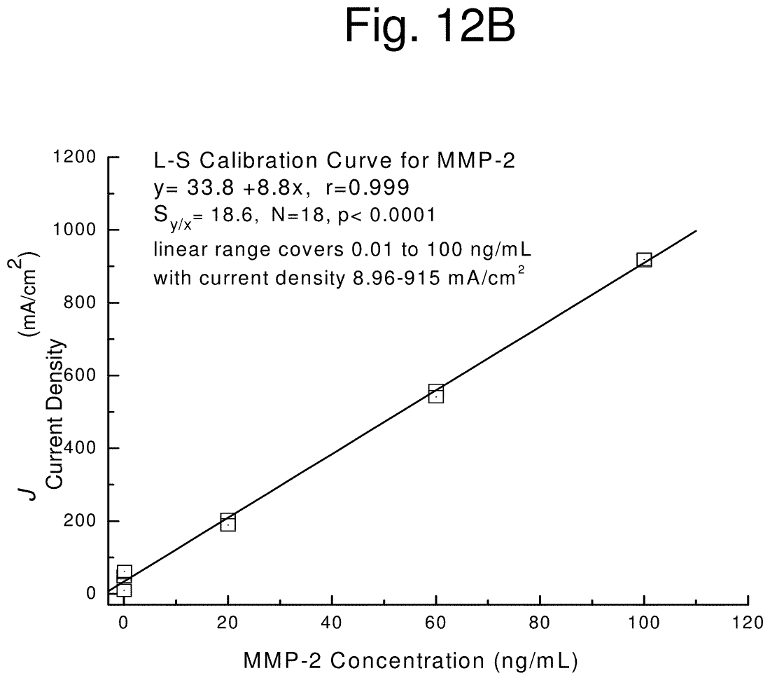

FIG. 12A depicts the CA curve profiles from 0.02 fg/mL to 40 fg/mL. The insert is the linear regression curve with over the same MMP2 range as in FIG. 12A. FIG. 12B depicts a linear calibration plot of the current density vs. MMP-2 concentration range from 0.01 ng/mL to 100 ng/mL with a linear regression equation Y=33.8+8.8X, r=0.999 (n=18), P<0.0001, Sy/x=18.6.

### Example 14—Results in the Quantitation of MMP-2 by the DSCPO Method

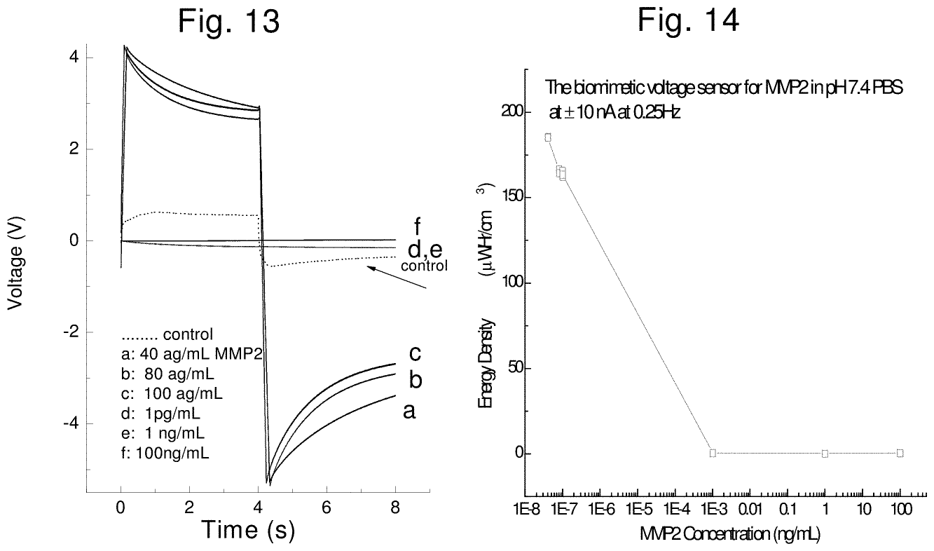

FIG. 13 depicts the DSCPO voltage curves vs. time at 0.25 Hz at ±10A over 40 ag/mL to 100 ng/mL MMP-2 concentrations against the control samples with each sample run triplicates. FIG. 14 depicts the volumetric energy density vs. MMP-2 concentrations, and it produced a similar impression value of 1.47% (n=18) over MMP2 concentration 40 ag/mL to 100 ng/mL over energy density between 185-0.47 μWHR/cm³.

### Example 15—Direct Measuring MMP-2 in NIST 965A Human Serum Specimens

The preliminary evaluation of the method application was conducted using the CA method to measure the MMP-2. The sensor was able to directly detect MMP2 in pure NIST serum specimens in the concentrations of 81.15±0.10 ag/mL for normal, 1.13±0.0016 pg/mL for hypoglycemia and 1.4±0.0001 pg/mL in hyperglycemia serum, respectively.

### Example 16—MMP-2 Concentration Levels Affect on the Sensor Energy Density Map

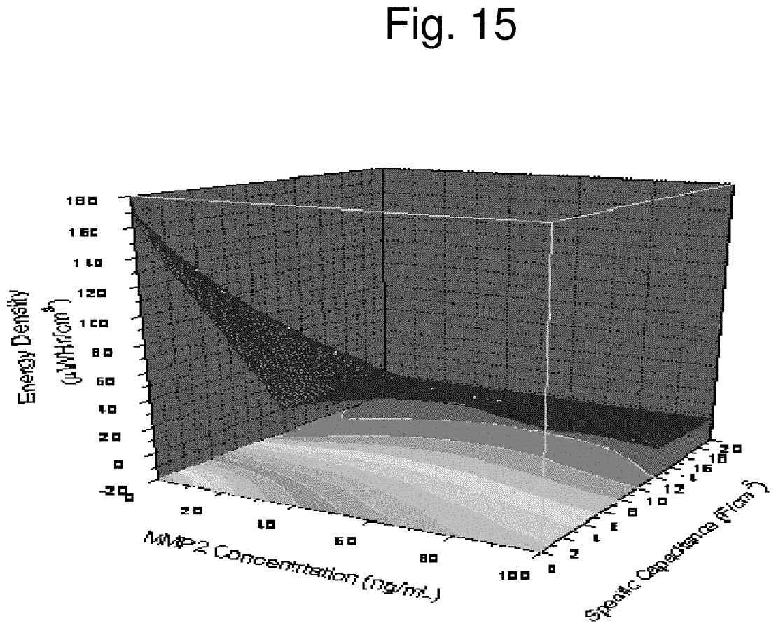

The DSCPO method was used to study the MMP-2 concentration level's affect on the sensor's energy density change related to specific capacitance change. The DSCPO results were obtained in the MMP-2 quantitation study described in the above section. The results were based on the equation of volumetric energy density, E=Cₛ·(ΔV)²/(2×3600), where Cₛ is the specific volumetric capacitance, Cₛ=[−i·Δt/ΔV]/L, Cₛ is in F/cm³ [21-22]. Δt is the time change in seconds, ΔV is the voltage change in V, i is the current in Amps, and L is the volume in cm³. FIG. 15 depicts a 3D map of the relationship between energy density of the sensor, MMP-2 concentration and specific capacitance using the voltage method. It was observed that lower MMP-2 concentration and lower specific capacitance are associated with higher energy density.

### CONCLUSION

We have demonstrated the memristive/memcapacitive device with vertex double-helical polarized biomimetic protein nanotubules forming a double membrane with potential gradient mimicking mitochondria's inner double membrane. We also observed the Fermi arcs on the surface of the nanostructured organic polymer membrane at the first time through a pair of polarized double-helical circular current flow to induce the Fermi arcs occurrence. The Fermi arcs promoted a direct chelating with zinc ions of the MMP-2 without any antibody, tracer, or reagent used at room temperature was accomplished. The observation of the pair of Dirac Cones became alignment and strengthened with each other in the presence of MMP-2 compared without MMP-2 may open new doors for medical doctors, scientists and engineers to pursue new types of devices and therapies in the future.

The MMP-2 can be detected with ag/mL level sensitivity and the DOL reached orders of magnitude lower than published reports with simplified procedures by two instrumental methods were also demonstrated using human serum specimens, and the DOL reached orders of magnitude lower than published reports under antibody-free, tracer-free, and reagent-free conditions with simplified procedures by two instrumental methods. The results show a feasible application for the development of commercial fast and real-time monitoring of MMPs devices for various diseases.

### REFERENCES

[1]. W. C. Parks and R. P. Mecham, Matrix metalloproteinases, Academic Press, San Diego, 1998.
[2] K. Siemianowicz, W. Likus and J. Markowski, Metalloproteinases in Brain Tumors, 2015. http://dx.doi.org/10.5772/58964.
[3]. E-M Schnaeker, R. Ossig, T. Ludwig et. al., Microtubule-dependent matrix metalloproteinases-2/matrix metalloproteinases-9 exocytosis: Prerequisite in human melanoma cell invasion, Cancer Research, 64, 8924-8931, 2004.
[4]. M. Rydlova, 1. Holubec jr., M. Ludvikova jr, D. Klfert Biological activity and clinical implications of the matrix metalloproteinases, Anticancer Research 28: 1389-1398, 2008.
[5]. F. Riedel, K. Gotte, J. Schuwalb, K. Hormann, Serum levels of matrix metalloproteinase-2 and -9 in patients with head and neck squamons cell carcinoma, Anticancer Research, 20(5A), 3045-3049, 2000.
[6]. K. Dahiya, R. Dhankhar, Updated overview of current biomarkers in head and neck carcinoma, World J. of Methodology, 6(1), 77-86, 2016.
[7]. S. Ghargozlian, K. Svennevig, H-J Bangstad et. al., Matrix metalloproteinases in subjects with type 1 diabetes, BMC Clinical Pathology, 9(7), 2009. doi:10.1186/1472-6890-9-7.
[8]. M. Hermandez-Guillamon, S. Mawhirt, S. Blais, et. al., Sequential beta-amyloidal degradation by the matrix metalloproteinases MMP-2 and MMP-9, J. of Biol Chem, 290(24), 15078-15091, 2015.
[9]. R Wang, G Q Zeng, R Z Tong, D Zhou, Z Hong, Serum matrix metalloproteinase-2: A potential biomarker for diagnosis of epilepsy, Epilepsy Research, 122, 114-119, 2016.
[10]. R E. Vandenbroucke C. Libert. Is there new hope for the therapeutic matrix metalloproteinase inhibition? Nature Reviews Drug Discovery, 13, 904-927, 2014.
[11]. G. Yang. L. Li, R K. Rana, Zhu. Assembled gold nanoparticles on nitrogen-doped graphene for ultrasensitive electrochemical detection of matrix metalloproteinase-2, Carbon 61, 367-366, 2013.
[12]. T. Zheng, R. Zhang, Q. Zhang et. al., Ultrasensitive dual-channel detection of matrix metalloproteinase-2 in human serum using gold-quantum dot core-satellite nanoprobes, ChemComm, 49, 7881-7883, 2016.
[13]. E. T. Chen, S-h Duh, C. Ngatchou, J. T. Thornton and P. T. Kissinger, Selectivity study for a reagent-less and enzyme-free nanopore glucose sensor against interferences from non-glucose sugars and other substances, NSTi-Nanotech (3), 101-104, 2011.
[14]. E. T. Chen, Y. Shen, C. Ngatchou, J. T. Thornton, S-h Duh, P. T. Kissinger, A nanopore biomimetic device quantitatively detects early stage cancer cells; a contour map multiple variable correlation method assesses the heat of cancer cells released, NSTi-Nanotech, 198-201, 2012.
[15]. E. T. Chen, J. T. Thornton, C. Ngatchou, S-h Duh, P. T. Kissinger, Study of the correlations between direct electron transfer rate constants and the effectiveness of cancer inhibitors using nanobiomimetic sensors, NSTi-Nanotech (3), 115-118, 2013.
[16]. S-H. Duh, J. Thornton, P. T. Kissinger and E. T. Chen, A nanobiomimetic neuronal memcapacitor serves as a voltage sensor and an amperometry sensor for reagent-less direct detection of sub pM soluble Amyloid-β. Biotech, Biomaterials and Biomedical: TechConnect Briefs, 172-175, 2015.
[17]. (a) E. T Chen, J. Thorten, C. Ngatchou, S-H Duh, Nanostructured memristor sensor mimics acetylcholinesterase (ACHE) active sites in the gorge for fM detection of acetylcholine, NSTi-Nanotech, 2, 169-172, 2014. (b) E. T. Chen, J. T. Thornton and Mulchi, Jr. C., Early forming a hummingbird-like hovering neural network circuitry pattern with reentrant spatio-temporal energy-sensory orientation privileged to avoid “epilepsy” based on a biomimetic acetylcholinesterase memcapacitor prosthesis, Sensors and Transducer. 191(8), 84-99, 2015. (c). Human milk shows immunological advantages over organic milk samples for infants in the presence of Lipopolysaccharide (LPS) in 3D energy maps using an organic nanobiomimetic memristor/memcapacitor, Sensors and Transducers, 203(8), 57-68, 2016.
[18]. E. T. Chen, J. Thornton and C. Mulchi Jr., Mapping circular current for a single brain cancer cell's spatial-temporal orientations based on a memristor/memcapacitor, Sensors & Transducers, 183(12), 72-83, 2014.
[19]. J. L. Malcos and W. O. Hancock, Engineering tubulin: microtubule functionalization approaches for nanoscale device applications, Appl. Microbiol Biotechnol, 90(1), 1-10, 2011.
[20]. J. T. Thornton, Jr. C. Mulchi, P. T. Kissinger and E. T. Chen, Acetylcholine Repairs the Amyloid-beta Damage on Brain Circuitry and Memory Loss From a “Mutated Biomimetic Acetylcholinesterase” Neuronal Memcapacitor During Slow-Wave Sleeping, Advanced Manufacturing, Electronics and Microsystems: TechConnect Briefs, 226-229, 2015.
[21]. E. T. Chen, J. T. Thornton and Jr. C. Mulchi, Early Forming a Hummingbird-like Hovering Neural network Circuitry Pattern with Reentrant Spatio-temporal Energy-Sensory Orientation Privileged to Avoid “Epilepsy” Based on a Biomimetic Acetylcholinesterase Memcapacitor Prosthesis, Sensors and Transducers Journal, 191(8), 84-99, 2015.
[22]. J. T. Thornton, Jr. C. Mulchi, P. T. Kissinger, E. T. Chen, In Vitro Restoration of an Amyloid-Beta Altered Network Circuitry in a ‘Mutated Biomimetic Acetylcholinesterase’ Memristor/Memcapacitor Neural Prosthesis, Sensors and Transducers Journal, 191(8), 100-113, 2015.
[23]. E. T. Chen, Nanostructured Biomimetic-Enzyme Sensing And Energy Storage, Dekker Encyclopedia of Nanoscience and Nanotechnology, First volume, Mar. 8, 2013.
Nanostructured Biomimetic Sensing And Energy Storage: Organic Memristor/Memcapacitors, Dekker Encyclopedia of Nanoscience and Nanotechnology, Third Edition, DOI:10.1081/E-ENN3-120054061, Jan. 18, 2017. www.crcnetbase.com
[24]. S-Y. Xu, I. Belopolski, N. Alidoust et. al., Discoveries of a weyl fermion semimetal and topological Fermi arcs, Science 349(6248), 613-617, 2015.
[25]. S-Y. Xu, C. Liu, S. K. Kushwaha, et. al., Observation of fermi arc surface in a topological metal: A new type of 2D electron gas, Science 347(6219), 294-298, 2015.
[26]. S-Y. Xu, N. Alidoust, I. Belopolski, et. al., Observation of a Weyl fermion state with fermi arcs in niobium arsenide, Nature Physics, 11, 748-754, 2015.
[27]. J. J. Yang, D. B. Strukov and D. R. Stewart, Memristive devices for computing, Nature Nanotechnology, 8, 13-24, 2013.
[28]. E. T. Chen and H. L. Pardue, Analytical applications of catalytic properties of modified cyclodextrins, Anal. Chem, 65(19), 2583-2587, 1993.
[29]. M. F. El-Kady, V. Strong, S. Dubin, R. B. Kaner, Laser Scribing of High-Performance and Flexible Graphene-Based Electrochemical Capacitors, supplement materials, Science, 335, 1326-1330, 2012.
[30]. J. R. Miller, R. A. Outlaw, B. C. Holloway, Graphene double-layer capacitor with ac line-filtering performance, Science, 329, 1637-1639, 2010.
[31]. O. Kleifeld, P. E. Van den Steen, A. Frenkel et. al., Structural characterization of the catalytic active site in the latent and active natural gelatinase B from human neutrophils, The journal of Biological Chem., 275(44), 34335-34343, 2000.
[32]. J. Pottel, E. Therrien, J. L. Gleason and N. Moitessier, Docking ligands into flexible and solvated macromolecules, 6. Development and application to the docking of HDACs and other zinc metalloenzymes inhibitors, J. of Chem information and modeling, 54, 254-265, 2014.
[33] M. D. Ventra, Y. V. Pershin, On the physical properties of memristive, memcapacitive, and meminductive systems, Nanotechnology, 24, 255201, 2013.
[34]. K. Conant, P. E. Gottschall, Matrix metalloproteinases in the central nerve system, Imperial College Press, 2005.
[35]. N. J. Clendeninn, K. Appelt, Matrix metalloproteinases inhibitors in cancer therapy, XIII, 262, A product of Humana Press, Springer, 2001.
[36]. E. D. Karagiannis, A. S. Popel, A theoretical model of type1 collagen proteolysis by matrix metalloproteinase (MMP)2 and membrane type 1 MMP in the presence of tissue inhibitor of metalloproteinase 2, The J. Biological Chemistry, 279(37), 39105-39114, 2004.
[37]. D. Rai, O. Hod and A. Nitzan, Magnetic field control of the current through molecular ring junctions, J. Physical Chemistry Letters, 2, 2118, 2011.
[38]. R. Islas, T. Heine and G. Merino, The induced magnetic field, Accounts of Chemical Research, 45(2), 215, 2012.
[39]. E. T. Chen, J. T. Thornton, and Jr. C. Mulchi, Mapping Circular Current for a Single Brain Cancer Cell's Spatial-Temporal Orientations Based on a Memristor/Memcapacitor, Sensors & Transducers, 183(12), 72-83, 2014,
[40]. G. M. Cooper and R. E. Hausman, The cell, A molecular approach, 7^{nd} edition, Boston University, Sunderland (Mass.), Sinauer Associates, 2015.
[41]. E. T. Chen, J. T. Thornton, S-H. Duh and P. T. Kissinger, Organic Nanobiomimetic Memristive/Memcapacitive Devices Ultrasensitive Direct Detect Matrix Matelloproteinase-2 in Human Serum, Biotech, Biomaterials and Biomedical, TechConnect Briefs, 271-274, 2017.

What is claimed is:

1. An Organic Memristive/Memcapacitive devices comprising of
(a) an electrode comprising a substrate of gold and on the top of the electrode affixed a self-assembling membrane (SAM) comprising a polymer matrix comprised of an electrically conductive copolymer; wherein the copolymer is further comprised of:
one or more imidazole substituted dimethyl-β-cyclodextrin (bM-β-DMCD) molecules; one or more β-cyclodextrin (β-CD) having at least one or more acetyl groups as TCD; one or more polyethylene glycol (PEG) polymers; one or more poly(4-vinylpyridine) (PVP) polymers;
(b) Cross-linked nanotubes made on the SAM promote direct electron-relay that mimicked a function of Matrix Metalloproteinase (MMP).

2. According to claim 1, wherein the SAM forms a vertex double-helical circular current in a 3D architecture through hydrogen bounding or hydrophobic interaction between TCD . . . PEG, TCD . . . PVP and bM-β-DMCD.

3. According to claim 2, wherein the double-helical circular current is a bidirectional direct electron-transfer (DET) circular current.

4. According to claim 3, wherein the circular current upon applied a potential possess a Fermi arc state glowing with nodes present.

5. According to claim 3, wherein the memristive/memcapacitive devices are dual function electrochemical devices for sensing and energy harvesting.

6. According to claim 1, wherein the sensor has the Detection of Limits (DOL) value of 8.67×10⁻¹⁸ g/mL in the PBS solution for direct quantitation of the MMP2 concentration between 20 ag/mL to 100 ng/mL with a Relative Pooled Standard Deviation 1.4% using a Chronoamperometric method.

7. According to claim 1, wherein the sensor has another function as a voltage sensor for detection of MMP-2 with an impression value of 1.47% over MMP2 concentration 40 ag/mL to 100 ng/mL over energy density between 185-0.47 μWHR/cm³.

8. According to claim 1, wherein the sensor direct detects MMP-2 in serum specimens in the concentrations of 81.15±0.10 ag/mL for normal glucose serum, 1.13±0.0016 pg/mL for hypoglycemia and 1.4±0.0001 pg/mL in hyperglycemia serum, respectively.

9. According to claim 1, wherein the device is an energy harvesting device, which spontaneously discharge at 50 mA for 16.5 hours with an energy density of 140 WHr/cm².

10. According to claim 1, wherein the device is under antibody-free, tracer-free, and reagent-free conditions.

11. According to claim 6, wherein the sensor direct detects protein in 4 ms without a need for sample preparation.

12. According to claim 11, wherein the sensor detects a protein without suffering interference.
